(19) United States
(12) Patent Application Publication
(10) Pub. No.: US 2009/0112298 A1
(43) Pub. Date: Apr. 30, 2009

(54) HIGH EFFICIENCY THERMAL ENERGY TRANSFER PAD

(75) Inventors: Joel T. Jusiak, Holland, NY (US); Karl H. Cazzini, Lindenhurst, IL (US)

Correspondence Address: KEVIN D. MCCARTHY;ROACH BROWN MCCARTHY & GRUBER, P.C., 424 MAIN STREET, 1920 LIBERTY BUILDING, BUFFALO, NY, 14202, US

(73) Assignee: GAYMAR INDUSTRIES, INC., Orchard Park, NY (US)

(21) Appl. No.: 12/258,895

(22) Filed: Oct. 27, 2008

Related U.S. Application Data
(60) Provisional application No. 60/984,096, filed on Oct. 31, 2007.

(51) Int. Cl. A61F 7/00

(52) U.S. Cl. 607/104

(57) ABSTRACT

A thermal energy transfer pad is disclosed. The thermal energy transfer pad has a first flexible, thermal energy transfer sheet and a second flexible, thermal energy transfer sheet. The first flexible, thermal energy transfer sheet (a) is made of a first fluid impervious material, (b) has a perimeter measurement of A prior to manufacturing and (c) has a first thermal energy transfer thickness. The first flexible, thermal energy transfer sheet is molded to form fluid path troughs defined by interior protuberances and the first sheet's perimeter edge. That molding alters the first flexible, thermal energy transfer sheet's perimeter measurement to B, which is less than A. The second thermal energy transfer sheet (a) is made of a second fluid impervious material, (b) has a perimeter measurement of B and (c) has a second thermal energy transfer thickness. The second thermal energy transfer sheet is sealed to the first thermal energy transfer sheet along the first sheet's perimeter and at the first sheet's interior protuberances. That sealing creates a tortuous fluid path in the fluid path troughs The resulting pad has a significantly (1) decreased chance of the fluid being occluded in the fluid path and (2) increased thermal energy transfer rate to the patient.

### CLAIM OF PRIORITY

This application claims priority to U.S. provisional patent application Ser. No. 60/984,096, filed on Oct. 31, 2007.

### FIELD OF THE INVENTION

The present invention is directed to thermal energy transfer pads.

### BACKGROUND OF THE INVENTION

It has become well known to treat bodily injuries, ailments and diseases by heating and/or cooling an affected body part or area. The application of heat and/or cold to an affected body part or area has been used to alleviate pain, accelerate healing, inhibit swelling or edema, reduce inflammation, reduce hematoma formation, improve flexibility and range of motion, decrease muscle spasm and restore strength. In particular, cold has been applied to an affected body part or area to slow down circulation and, therefore, the flow of blood to the affected body part or area, slow enzyme function and metabolic reactions, retard metabolism within tissue cells, contract blood vessels and block nerve impulses. The application of heat to an affected body part or area has been found to diminish pain impulses, increase collagen elasticity, accelerate cellular metabolism, dilate blood vessels, increase circulation and speed up the rate of enzymatic reactions. Injuries, ailments and diseases involving soft tissue, muscles, ligaments, tendons and/or joints have been effectively treated with heat and/or cold therapy. The application of heat and/or cold to a human body has also been used to treat hypothermia and hyperthermia and to alter or maintain core body temperature.

### Gaymar's T/Pad

The application of heat and/or cold to the human body can be accomplished by thermal energy transfer pads. Thermal energy transfer pads define an arrangement of fluid channels therein for continuously circulating a thermal transfer fluid within the pads. These pads have been used for localized heating and/or cooling of affected parts of the human body for many years. In particular, Gaymar Industries, Inc., the assignee of this application, manufactures thermal energy transfer pads and has done so since the 1970's. Gaymar's thermal energy transfer pads are known as T/Pads. These pads receive a fluid, allow the fluid to circulate within the interior of the pad, and release the fluid to a fluid source or fluid receiver through an outlet conduit, and/or ambient air if there are apertures spaced throughout the pad and the fluid is a gas.

In particular, Gaymar's thermal energy transfer pad has two flexible, thermal energy transfer sheets of (a) fluid impervious material (polyurethane; polyvinyl chloride; polypropylene, and/or nylon), (b) similar perimeter measurements at all times and (c) similar thicknesses (0.0015 to 0.0040 inches thick).

The transfer pad also has an edge seal. The edge seal connects the two sheets to one another continuously along the perimeter to form a fluid receiving cavity between the sheets. At least one or a plurality of inner seals or seams is disposed interiorly of the edge seal at which the sheets are connected to one another to form a tortuous fluid passage(s) in the thermal energy transfer pad. This product design is a conventional dual bump thermal pad system, as illustrated at FIG. 6. A dual bump pad system has both sides bumpy—which is avoided in the present invention.

The fluid can be a liquid and/or gas at a desired temperature. The temperature can be controlled, for example, by a Medi-Therm II fluid temperature control device, a T-Pump fluid temperature control device, or equivalents thereof. Those temperature controlling devices push the fluid through the fluid passage. If those devices pulled (negative pressure) the fluid through the fluid passage, the devices would cause the fluid passages to collapse between the internal seams and seals. Once the fluid passages collapse, the fluid is unable to circulate in the fluid passages and effectively transfer the fluid's thermal energy to the patient.

### Carson's Device

In a desire to use negative pressure, Carlson discloses in U.S. Pat. No. 6,375,674, thermal transfer pads that are variations of Gaymar's transfer pad. Carlson's device has a flexible thermal energy transfer sheet that (a) contacts the patient's skin and (b) has an edge seal. The edge seal connects the flexible thermal transfer sheet to “an insulating, flexible base sheet having projections” continuously along the perimeter to form a fluid receiving cavity between the materials.

The projections are solid objects made from the same material as the insulating, flexible base material and extend from the interior surface of the insulating, flexible base sheet toward the flexible thermal energy transfer sheet. Carlson clearly states the flexible thermal energy transfer sheet does not have to be sealed to the projections' apex (area closest to the flexible thermal energy transfer sheet) and is vague about an alternative embodiment. Sealing the flexible thermal energy transfer sheet to the projections' apex is unnecessary because (1) a fluid path is defined in the valleys between the projections, (2) the negative pressure applied to pull the fluid through the valleys also pulls the thermal energy transfer sheet toward the projections, and (3) the projections “support” the thermal energy transfer sheet from collapsing into the valleys which would inhibit the fluid path from having any type of occlusion.

In particular, Carlson illustrates the insulating base sheet's exterior surface is planar and has no undulation and/or curvatures that correspond to any projection. That confirms the base material having projections is an insulation material designed to “inhibit the heat transfer between the surrounding air and the fluid circulated through the fluid containing layer thereby enhancing the efficiency of the pad” to transfer thermal energy only through the flexible thermal energy transfer sheet.

### SUMMARY OF THE INVENTION

A thermal energy transfer pad is disclosed. The thermal energy transfer pad has a first flexible, thermal energy transfer sheet and a second flexible, thermal energy transfer sheet. The first flexible, thermal energy transfer sheet (a) is made of a first fluid impervious material, (b) has a perimeter measurement of A prior to manufacturing and (c) has a first thermal energy transfer thickness. The first flexible, thermal energy transfer sheet is molded to form fluid path troughs defined by interior protuberances and the first sheet's perimeter edge. That molding alters the first flexible, thermal energy transfer sheet's perimeter measurement to B, which is less than A. The second thermal energy transfer sheet (a) is made of a second fluid impervious material, (b) has a perimeter measurement of B and (c) has a second thermal energy transfer thickness. The second thermal energy transfer sheet is sealed to the first thermal energy transfer sheet along the first sheet's perimeter and at the first sheet's interior protuberances. That sealing creates a tortuous fluid path in the fluid path troughs. The resulting pad has a significantly (1) decreased chance of the fluid being occluded in the fluid path and (2) increased thermal energy transfer rate to the patient.

### BRIEF DESCRIPTION OF THE FIGURES

FIG. 1 illustrates a top view of the present invention—a thermal energy transfer pad.

FIG. 2 illustrates an enlarged cross-sectional view from box 28 of FIG. 1 along lines 2-2.

FIG. 3 illustrates a schematic of manufacturing process to fabricate the thermal energy transfer pad.

FIG. 4 illustrates an alternative embodiment of FIG. 2 that is configured about a convex-like surface (not shown).

FIG. 5 illustrates an alternative embodiment of FIG. 2 that is configured about a concave-like surface (not shown).

FIG. 6 illustrates a conventional prior art dual bump thermal pad device's surface area that contacts a patient.

FIG. 7 illustrates the claimed invention's surface area that contacts a patient.

FIG. 8 is a chart illustrating the heat flux over time of the two disclosed prior art embodiments and the claimed invention.

### DETAILED DESCRIPTION OF THE INVENTION

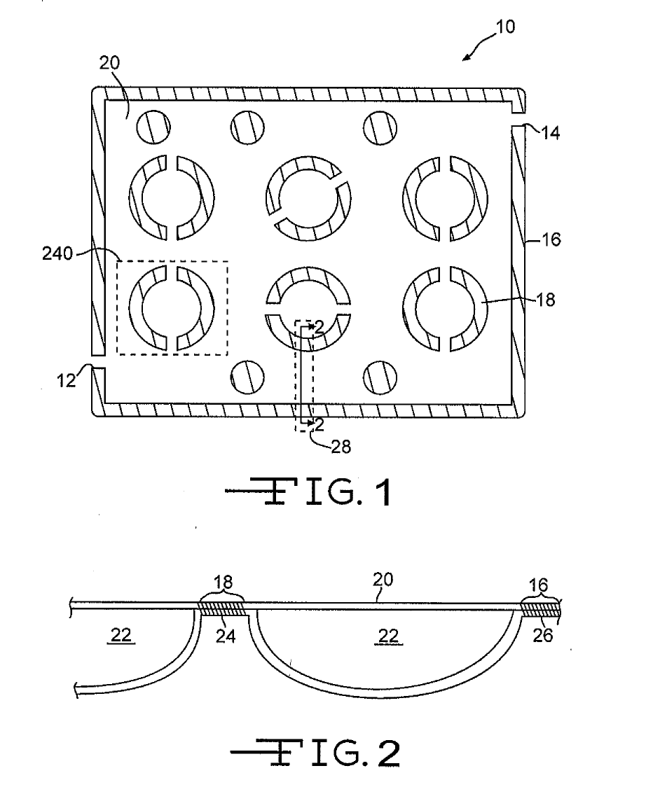

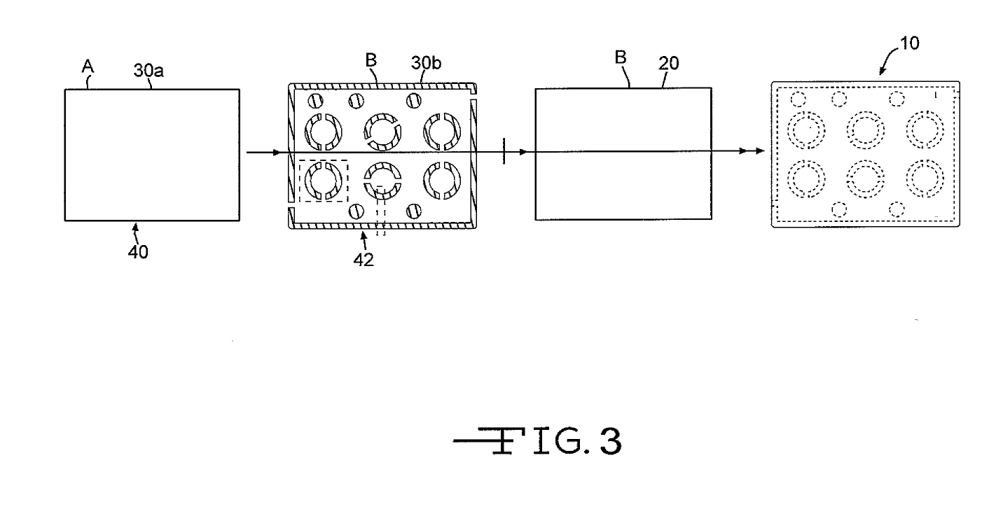

A thermal energy transfer pad 10 is illustrated in FIGS. 1 and 2. The thermal energy transfer pad 10 has a first flexible, thermal energy transfer sheet 30 (illustrated in FIG. 2) and a second flexible, thermal energy transfer sheet 20 (illustrated in FIGS. 1 and 2).

To appreciate this invention in greater detail, we will revert to the process to manufacture the claimed invention's thermal energy transfer pad 10 that is illustrated in FIG. 3.

The first step requires obtaining the first flexible, thermal energy transfer sheet 30. The first flexible, thermal energy transfer sheet 30 is made of a first fluid impervious material. Examples of that material include polyurethane; polyvinyl chloride; polypropylene, mylar (with or without a polymeric resin material thereon) and/or nylon. The first flexible thermal energy transfer sheet 30 also has a first thermal energy transfer thickness. That thickness is sufficient to allow the fluid's thermal energy to pass through the sheet 30 without significant thermal energy decrease when it passes through. That thickness ranges from 0.0015 to 0.0040 inches. In other words, the first flexible thermal energy transfer sheet 30 has a thickness that is not an insulator. The transfer sheet 30 also has a perimeter measurement of A.

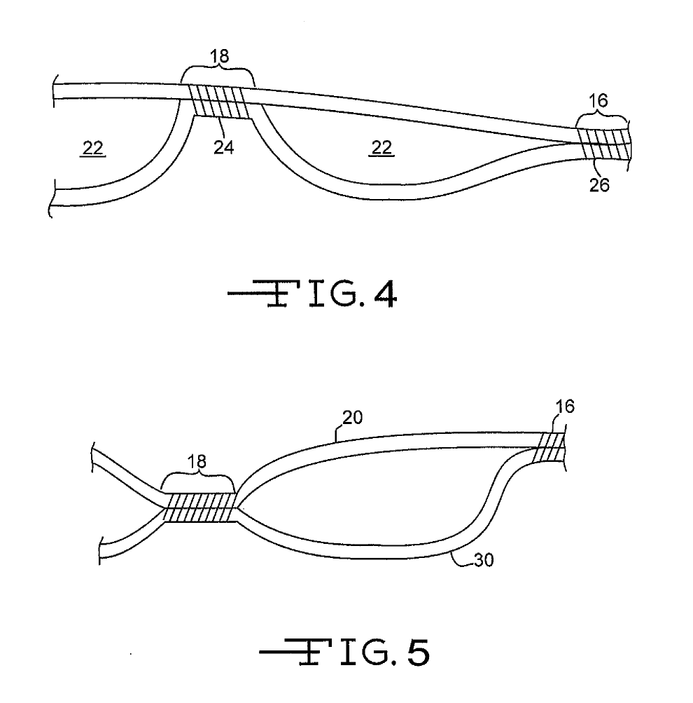

In FIG. 3, the first flexible thermal energy transfer sheet having a perimeter measurement of A is identified as item 30A. The first flexible, thermal energy transfer sheet 30A is molded by injection molding, vacuum forming or compression molding to form fluid path troughs 22 defined by interior protuberances 24 and the perimeter edge 26, as illustrated in FIG. 2. When formed into pad 10, the perimeter edge 26 can be in the same plane as the apex of the interior protuberance 24 as illustrated in FIG. 2, or an alternative plane as illustrated in FIG. 4. Reverting to FIG. 3, after the molding process, the first flexible, thermal energy transfer sheet 30A has a perimeter of about B (B is smaller than A), and is therefore identified as first flexible, thermal energy transfer sheet 30B.

The second flexible, thermal energy transfer sheet 20 is made of a first fluid impervious material. Examples of that material include polyurethane; polyvinyl chloride; polypropylene, mylar (with or without a polymeric resin material thereon) and/or nylon. The second flexible thermal energy transfer sheet 20 also has a second thermal energy transfer thickness. That thickness is sufficient to allow the fluid's thermal energy to pass through the sheet 20 without significant thermal energy decrease when it passes through. That thickness ranges from 0.0015 to 0.0040 inches. The transfer sheet 20 also has a perimeter measurement of B.

At the interior protuberances 24 and the perimeter edge 26, the first flexible, thermal energy transfer sheet 30B seals to the second flexible, thermal energy transfer sheet 20 to form (a) interior seals 18 and perimeter seals 16 and (b) the improved thermal energy transfer pad 10 having an inlet 12 and an outlet 14. The interior seals 18 and perimeter seals 16 define a tortuous fluid path. The tortuous fluid path defined by troughs 22 have a decreased chance of occluding the fluid.

The chance of occlusion is decreased because the trough 22 ensures the portions of the first flexible, thermal energy transfer sheet 30B and the second flexible, thermal energy transfer sheet 20 that define the tortuous fluid path are a desired distance from each other. Unlike the prior art, the interior protuberances 24 do not support the second flexible, thermal energy transfer sheet 20. The pad 10 is also unable to be subject to negative pressure. If negative pressure was used to pull the fluid through pad 10, the first flexible, thermal energy transfer sheet 30B and the second flexible, thermal energy transfer sheet 20 would collapse and occlude the fluid path. That teaching confirms the interior protuberances 24 do not support any sheet 20, 30. That teaching also confirms the current invention is limited to a positive pressure flow of fluid through the fluid path.

Another aspect of the current invention is that the interior protuberances 24 can be manipulated to different planes while retaining a fluid path trough configuration. An example of such manipulation is illustrated by comparing FIGS. 2 (contacting a planar surface), 4 (contacting a convex-like surface), and 5 (contacting a concave-like surface). That ability to manipulate the pad's shape, use of either side (preferably the planar surface), and retain the fluid path trough is a significant improvement over the prior art.

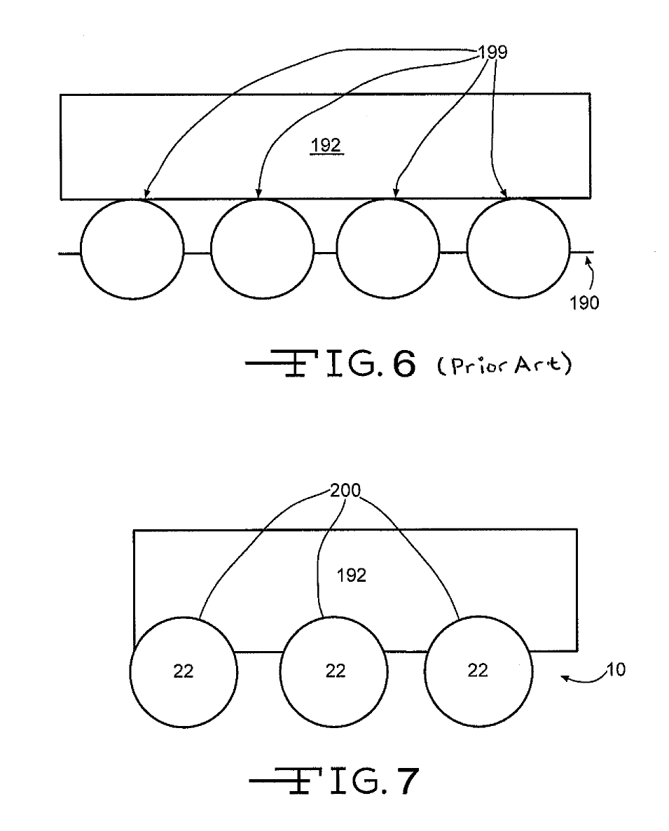

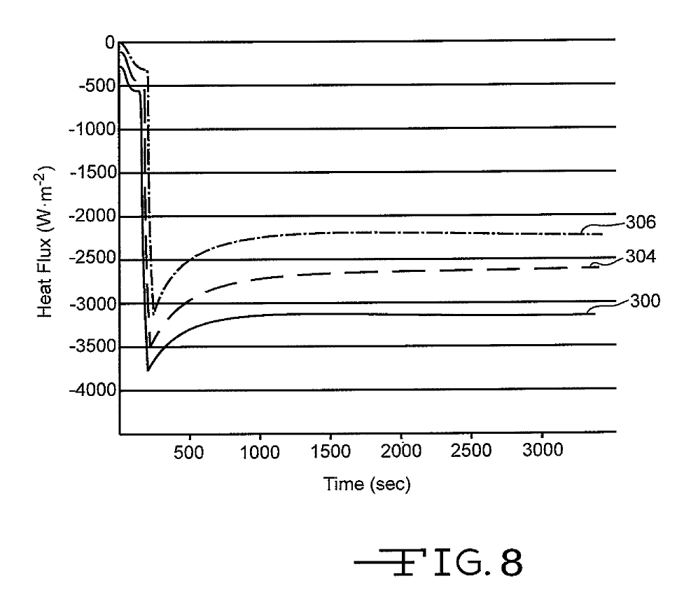

Other improvements include and are not limited to an increased area that contacts the patient compared to the prior art devices. In the prior art dual bump configuration, the surface area is significantly less because the prior art pad 190 contacts the patient 192 at a contact point 199 of each trough. See FIG. 6. In contrast, the present invention has surface contacting areas 200 that maximize the potential surface area that contacts the patient's body—see FIG. 7. The surface contacting areas 200 are significantly larger than all identified prior art. At chart 1, the applicants convey their measurement percentages of the surface area of Gaymar's T-Pad thermal device, Carlson's thermal device, and the claimed invention's thermal pad device that contacts a patient's skin. Those percentages are as follows:

CHART 1

| | Percentage of Surface Area that |
|---|---|
| Product Name | Contacts Patient |
| Gaymar's T-Pad (Prior Art) | 79.18% |
| Carlson Embodiment (Prior Art) | 89.23% |
| Claimed Invention | 92.06% |

The increased percentage of surface area that contacts the patient is only one of many improvements over the prior art thermal therapy devices. The increased surface area that contacts the patient is caused by (1) the flat, planar contacting surface 20 joined together to (2) the vacuum formed bumpy material 30b that forms the troughs 22. The current inventions troughs 22 are more flexible than the prior art. The flat planar surface combined with the vacuum formed bumpy material promotes that flexibility and application to a patient's various shapes and sizes.

Another improvement is in the heat flux of the thermal pad. Heat flux or thermal flux is a flow of energy per unit of area per unit of time. In SI units, it is measured in [W·m⁻²]. It has both a direction and a magnitude so it is a vectorial quantity. A desired heat flux value for a thermal pad is a lower negative heat flux value which means more thermal energy from the fluid in the thermal pad is being transmitted to the patient. In other words, a thermal pad having a −3200 W·m⁻² value is superior to a thermal pad having a −2800 W·m⁻² value. FIG. 8 illustrates the measurements of Gaymar's T-Pad, Carlson's pad and the present invention.

The Flux Heat measurements were taken by a conventional process of inserting the respective thermal pad in an insulation chamber. Each thermal pad was interconnected to Gaymar's Medi-therm hypo/hyper thermia device. The Medi-therm hypo/hyper thermia device delivered water (positively to the claimed invention; negatively to the Carlson embodiment since positive pressure would create a balloon effect; and positively or negatively through Gaymar's standard T-Pad device) at a predetermined temperature (41° C. and 5° C.) to each thermal pad positioned in the insulation chamber. Positioned on identical or essentially similar locations for each thermal pad were sensors that measured the thermal energy that passed from the fluid in the thermal pad through the thermal pad to the sensors. Each Heat Flux measurement was compared against a control to ensure the control exhibited the statistically similar Heat Flux results for each Heat Flux measurement. The average Heat Flux measurements for the claimed invention, the Carlson embodiment and Gaymar's standard T-Pad device are set forth in Chart 2.

CHART 2

| Product Name | Heat Flux Value | FIG. 8 Line |
|---|---|---|
| Gaymar's T-Pad (Prior Art) | −2300 Wm⁻² | 306 |
| Carlson Embodiment (Prior Art) | −2800 Wm⁻² | 304 |
| Claimed Invention | −3200 Wm⁻² | 300 |

As clearly illustrated in FIG. 8 and Chart 2, the claimed invention is significantly superior to the prior art in transferring the fluid's thermal energy to the patient. That transfer of heat capability with the increased surface area that is created by the novel manufacturing process to create a thermal pad are critical features of the claimed invention.

The significantly greater transfer of thermal energy is attributed to the increased area of contact surface on the patient due to one surface being planar during the manufacturing process, the other surface in molded to form troughs during the manufacturing process, and the ability of the two surfaces to be manipulated to obtain the desired increased surface contact; and the troughs that are not occluded even when the pad is manipulated into different shapes.

Internal protuberances 24 having a shape of a circle with internal breaks (a.k.a. portion of the letter C with a mirror image thereof and separated by a predetermined distance) 240, as illustrated in FIG. 1, have been determined to maximize the thermal energy transfer in the pad 10. Obviously, other internal protuberance 24 can exist in pad 10 but the circle with internal breaks design works well in the current invention to obtain the desired thermal transfer efficiency. It is believed the circle with internal breaks 240 design increases the area in which the fluid contacts the first and second sheets 20, 30. That increase in area in combination with the other embodiments of the present invention makes the thermal energy transfer pad more efficient in transferring thermal energy from the pad to the patient.

Alternatively, the first flexible, thermal energy transfer sheet 30 and/or the second flexible, thermal energy transfer sheet 20 can have a reflective thermal energy directing property thereon. The reflective thermal energy directing material, for example aluminum, is thermally bonded to the respective sheet 20, 30. Depending on the location of the reflective thermal energy directing material, the reflective thermal energy directing material can shield the patient from excess thermal energy or amplify the thermal energy applied to the patient by reflecting the thermal energy toward the patient.

The foregoing is a description of one presently preferred embodiment of our invention. The design parameters have been set out in a manner which we believe is understandable. Variations of designs can be made changing this particular preferred embodiment in major and minor manners without departing from the spirit and scope of our invention.

We claim:

1. A thermal energy transfer pad comprising
a first flexible, thermal energy transfer sheet (a) made of a first fluid impervious material, (b) molded to form fluid path troughs defined by interior protuberances and the first sheet's perimeter edge and (c) has a first thermal energy transfer thickness;
a second flexible, thermal energy transfer sheet (a) made of a second fluid impervious material, (b) is planar, (c) has a second thermal energy transfer thickness and (d) having a second perimeter edge wherein the second perimeter edge seals to the first sheet's perimeter edge after it has been molded to form the fluid path troughs and portions of the second interior sheet seals to the interior protuberances to form a tortuous fluid path.

2. The thermal energy transfer pad of claim 1 wherein material for the first flexible, thermal energy transfer sheet is selected from the group consisting of polyurethane, polyvinyl chloride, polypropylene, mylar, nylon; polyurethane with a reflective thermal energy directing material, polyvinyl chloride with a reflective thermal energy directing material, polypropylene with a reflective thermal energy directing material, mylar with a reflective thermal energy directing material and/or nylon with a reflective thermal energy directing material.

3. The thermal energy transfer pad of claim 2 wherein the material for the second flexible, thermal energy transfer sheet is the same as the first flexible, thermal energy transfer sheet.

4. The thermal energy transfer pad of claim 2 wherein the material for the second flexible, thermal energy transfer sheet is (a) different from the first flexible, thermal energy transfer sheet, and (b) selected from the group consisting of polyurethane; polyvinyl chloride; polypropylene, and/or nylon.

5. The thermal energy transfer pad of claim 1 wherein the material for the first thermal energy transfer thickness ranges from 0.0015 to 0.0040 inches.

6. The thermal energy transfer pad of claim 1 wherein the material for the second thermal energy transfer thickness ranges from 0.0015 to 0.0040 inches.

7. The thermal energy transfer pad of claim 1 wherein the interior protuberances include a circle with internal breaks design.

8. The thermal energy transfer pad of claim 1 wherein the molding is selected from the group consisting of injection molding, vacuum forming and compression molding.

9. The thermal energy transfer pad of claim 1 wherein the thermal energy transfer pad has an inlet and an outlet for fluid to pass through the tortuous fluid path.

10. The thermal energy transfer pad of claim 9 wherein the fluid is pushed into the inlet and out of the outlet by positive pressure, not pulled into the inlet and our the outlet by negative pressure.

11. A process to manufacture a thermal energy transfer pad comprising
obtaining a first flexible, thermal energy transfer sheet (a) made of a first fluid impervious material, (b) having a perimeter measurement of A and (c) has a first thermal energy transfer thickness;
molding the first flexible, thermal energy transfer sheet to form fluid path troughs defined by interior protuberances and the first sheet's perimeter edge, and as a result the first flexible, thermal energy transfer sheet has a perimeter measurement of B which is less than A;
sealing a planar second flexible, thermal energy transfer sheet (a) made of a second fluid impervious material, (b) having a perimeter measurement of B, (c) has a second thermal energy transfer thickness and (d) having a second perimeter edge to the molded first flexible, thermal energy transfer sheet wherein the second perimeter edge seals to the first sheet's perimeter edge and portions of the second interior sheet seals to the interior protuberances to form a tortuous fluid path.

12. The process of claim 11 wherein material for the first flexible, thermal energy transfer sheet is selected from the group consisting of polyurethane, polyvinyl chloride, polypropylene, mylar, nylon; polyurethane with a reflective thermal energy directing material, polyvinyl chloride with a reflective thermal energy directing material, polypropylene with a reflective thermal energy directing material, mylar with a reflective thermal energy directing material and/or nylon with a reflective thermal energy directing material.

13. The process of claim 12 wherein the material for the second flexible, thermal energy transfer sheet is the same as the first flexible, thermal energy transfer sheet.

14. The process of claim 12 wherein the material for the second flexible, thermal energy transfer sheet is (a) different from the first flexible, thermal energy transfer sheet, and (b) selected from the group consisting of polyurethane; polyvinyl chloride; polypropylene, and nylon.

15. The process of claim 11 wherein the material for the first thermal energy transfer thickness ranges from 0.0015 to 0.0040 inches

16. The process of claim 11 wherein the material for the second thermal energy transfer thickness ranges from 0.0015 to 0.0040 inches.

17. The process of claim 11 wherein the interior protuberances include a circle with internal breaks design.

18. The process of claim 11 wherein the molding is selected from the group consisting of injection molding, vacuum forming and compression molding.

19. The thermal energy transfer pad of claim 11 wherein the thermal energy transfer pad has an inlet and an outlet for fluid to pass through the tortuous fluid path.

20. The thermal energy transfer pad of claim 19 wherein the fluid is pushed into the inlet and out of the outlet by positive pressure, not pulled into the inlet and our the outlet by negative pressure.
